(19) United States
(12) Patent Application Publication
(10) Pub. No.: US 2024/0207043 A1
(43) Pub. Date: Jun. 27, 2024

(54) MODULAR IMPLANT WITH STABILIZATION

(71) Applicant: Alcon Inc., Fribourg (CH)

(72) Inventors: William Jacob Spenner Dolla, Plano, TX (US); Kamal K. Das, Arlington, TX (US)

(21) Appl. No.: 18/527,510

(22) Filed: Dec. 4, 2023

Related U.S. Application Data
(60) Provisional application No. 63/476,403, filed on Dec. 21, 2022.

(51) Int. Cl. A61F 2/16

(52) U.S. Cl. CPC A61F 2/1648; A61F 2/1629; A61F 2002/169

(57) ABSTRACT

A modular optical implant, which may comprise a base ring and an optic with features that enable assembly, alignment, and stabilization of adjunct optics. The optic may have features that interface with other optics and improve optical outcomes through rigid alignment of adjunct optics, mitigation of anterior capsule opacification, in vivo identification of optical features while implanting the lens, and reduction of refractive error. More generally, some embodiments may be an optical implant, which may comprise a posterior optic surface, an anterior optic surface, an optic edge between the posterior optic surface and the anterior optic surface, a locking tab coupled to the posterior optic surface, and an optic stabilizer coupled to the anterior optic surface.

### The invention set forth in the appended claims relates generally to surgical implants, such as ophthalmic lenses, including, without limitation, intraocular lenses.

### BACKGROUND

The human eye can suffer various maladies causing mild deterioration to complete loss of vision. While contact lenses and eyeglasses can compensate for some ailments, ophthalmic surgery may be required for others. In some instances, implants may be beneficial or desirable. For example, an intraocular lens may replace a clouded natural lens within an eye to improve vision.

While the benefits of intraocular lenses and other implants are known, improvements to lenses, delivery systems, components, and processes continue to improve outcomes and benefit patients.

### BRIEF SUMMARY

New and useful systems, apparatuses, and methods for eye surgery are set forth in the appended claims. Illustrative embodiments are also provided to enable a person skilled in the art to make and use the claimed subject matter.

For example, some embodiments may comprise a modular intraocular lens that can facilitate implantation and improve mechanical stability, usability, and efficacy. Some embodiments may include a circumferentially locking optic with anterior features that can enhance modularity. For example, such a modular lens may comprise a base ring and an optic with features that enable assembly, alignment, and stabilization of adjunct optics. In some examples, the optic may have features that interface with other optics and improve optical outcomes through rigid alignment of adjunct optics, mitigation of anterior capsule opacification, in vivo identification of optical features while implanting the lens, and reduction of refractive error.

More generally, some embodiments may be an optical implant, which may comprise a posterior optic surface, an anterior optic surface, an optic edge between the posterior optic surface and the anterior optic surface, a locking tab coupled to the posterior optic surface, and an optic stabilizer coupled to the anterior optic surface. In more particular embodiments, the optic stabilizer may comprise an angular protrusion from the anterior optic surface. The optic stabilizer may comprise an adjunct support and an optic seat in some examples. Additionally, or alternatively, some embodiments of the optic stabilizer may comprise a stub or post protruding from the anterior surface. In yet other embodiments, the optical implant may additionally comprisc a base configured to be coupled to the locking tab. For example, the base may comprise a ring having an anterior surface, a posterior surface, an inner surface, and an outer surface; and a haptic coupled to the outer surface. The haptic can form a junction with the ring, and the locking tab can be configured to be positioned in the junction. Additionally, or alternatively, some embodiments may comprise an adjunct optic coupled to the optic stabilizer.

Other example embodiments of an optical implant may comprise a base and a primary optic. The primary optic may comprise a posterior optic surface, an anterior optic surface, an optic edge between the posterior optic surface and the anterior optic surface, a sidewall, a transition region between the posterior optic surface and the sidewall, locking tabs coupled to the posterior optic surface, a first optic stabilizer coupled to the anterior optic surface, and a second optic stabilizer coupled to the anterior optic surface. The locking tabs and the sidewall can be configured to overlap the base. In more particular example embodiments, the second optic stabilizer may be disposed opposite the first optic stabilizer. Some embodiments of the base may comprise a ring having an anterior surface, a posterior surface, an inner surface, and an outer surface; and a pair of haptics coupled to the outer surface. For example, some embodiments of the haptics may form junctions with the ring, and the locking tabs may be configured to be positioned in the junctions. Additionally, or alternatively, some embodiments may comprise an adjunct optic coupled to the first optic stabilizer and the second optic stabilizer.

In some example embodiments, the base may define a first axis, and the primary optic may define a second axis. The locking tabs may be aligned with the second axis in some embodiments and positioning the locking tabs in the junction can align the second axis with the first axis. For example, the base may comprise openings that define the first axis, and the primary optic may comprise orientation markers that define the second axis.

In yet other example embodiments, a method for assembling an optical implant may comprise providing a base, a primary optic, and an adjunct optic, wherein the primary optic comprises a locking tab, a sidewall, and an optic stabilizer. The primary optic may be positioned on the base so that sidewall overlaps at least a portion of the base. The primary optic may be rotated relative to the base to couple the locking tab to the base, and the adjunct optic may be coupled to the optic stabilizer. For example, in some embodiments, the base may comprise a ring having an anterior surface, a posterior surface, an inner surface, and an outer surface; and a haptic coupled to the outer surface. The haptic may form a junction with the ring, and rotating the primary optic may position the locking tab in the junction. Additionally, or alternatively, the base may further comprise openings that define a first axis, the primary optic may further comprise orientation markers that define a second axis, the locking tab may be aligned with the second axis, and rotating the primary optic can align the first axis and the second axis.

Features, elements, and aspects described in the context of some embodiments may also be omitted, combined, or replaced by alternative features. Other features, objectives, advantages, and a preferred mode of making and using the claimed subject matter are described in greater detail below with reference to the accompanying drawings of illustrative embodiments.

### BRIEF DESCRIPTION OF THE DRAWINGS

The accompanying drawings illustrate some objectives, advantages, and a preferred mode of making and using some embodiments of the claimed subject matter. Like reference numbers represent like parts in the examples.

FIG. 1 is an isometric view of an example optical implant.

FIG. 2 is an assembly view of the optical implant of FIG. 1.

FIG. 3 is an isometric view of an example of a base that may be associated with the optical implant of FIG. 1.

FIG. 4 is an isometric view of a primary optic that may be associated with the optical implant of FIG. 1.

FIG. 5 is a section view of the primary optic of FIG. 4 taken along line 5-5.

FIG. 6 is a side view of the primary optic of FIG. 4.

FIG. 7 is a front view of the optical implant of FIG. 1.

FIG. 8 is an isometric view of another example of an optical implant.

FIG. 9 is a front view of the optical implant of FIG. 8.

FIG. 10 is an isometric view of another example of an optical implant.

FIG. 11 is an isometric view of another example of an optical implant.

FIG. 12 is an isometric view of another example of a primary optic.

FIG. 13 is an isometric view of another example of an optical implant.

### DESCRIPTION OF EXAMPLE EMBODIMENTS

The following description of example embodiments provides information that enables a person skilled in the art to make and use the subject matter set forth in the appended claims, but it may omit certain details already well known in the art. The following detailed description is, therefore, to be taken as illustrative and not limiting.

The example embodiments may also be described herein with reference to spatial relationships between various elements or to the spatial orientation of various elements depicted in the attached drawings. In general, such relationships or orientation assume a frame of reference consistent with or relative to a patient in a position to receive an implant. However, as should be recognized by those skilled in the art, this frame of reference is merely a descriptive expedient rather than a strict requirement.

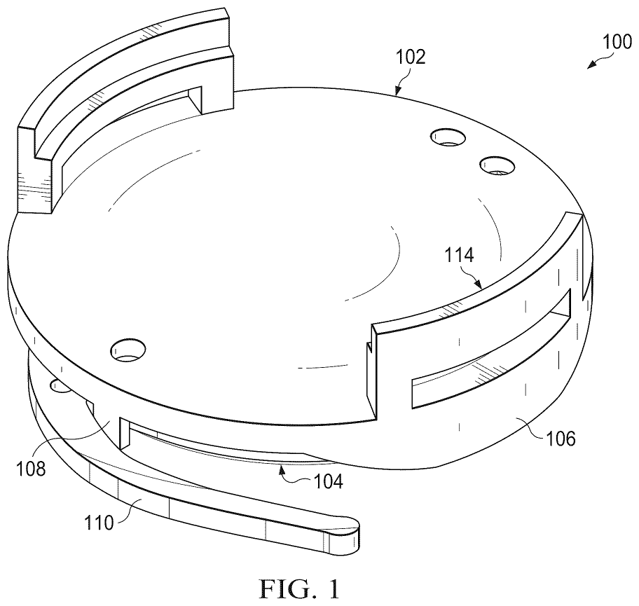

FIG. 1 is an isometric view of an optical implant 100, illustrating various features that may be associated with example embodiments suitable for implanting into an eye. As illustrated in FIG. 1, some embodiments of the optical implant 100 may include modular components that can be combined in various configurations. For example, the optical implant 100 may comprise an ophthalmic lens, such as a primary optic 102, seated anterior to a base 104. FIG. 1 further depicts a sidewall 106 on the primary optic 102. The primary optic 102 may additionally have one or more tabs 108, which may be configured to be coupled to the base 104. The sidewall 106 and the tabs 108 can overlap at least a portion of the base 104 to facilitate the positioning of the primary optic 102 on the base 104 or orienting the primary optic 102 relative to the base 104. If the primary optic 102 is positioned on the base 104, the sidewall 106 and the tab 108 can reduce or even prevent tilt and decentration of the primary optic 102. In some examples, the base 104 may also comprise one or more haptics 110.

FIG. 1 also illustrates an example of the primary optic 102 having one or more optic stabilizers 114. As shown in the example of FIG. 1, some embodiments of the optic stabilizers 114 may comprise angular protrusions extending from the primary optic 102.

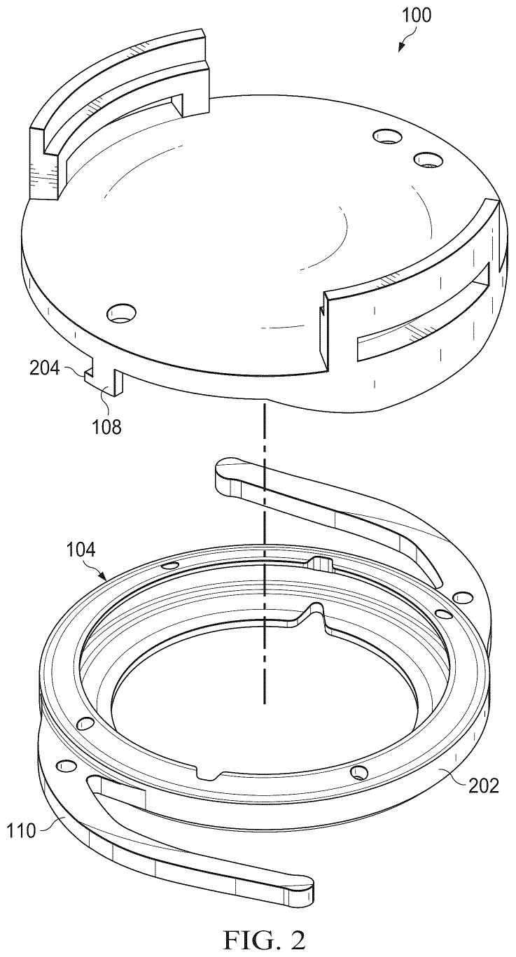

FIG. 2 is an assembly view of the optical implant 100 of FIG. 1, illustrating additional details that may be associated with some embodiments. In FIG. 2, for example, the base 104 comprises a ring 202, which may be coupled to the haptics 110. Additionally, or alternatively, the tabs 108 of the primary optic 102 may comprise or be coupled to an extension 204, which may be disposed posterior to the haptics 110 if assembled as shown in the example of FIG. 1.

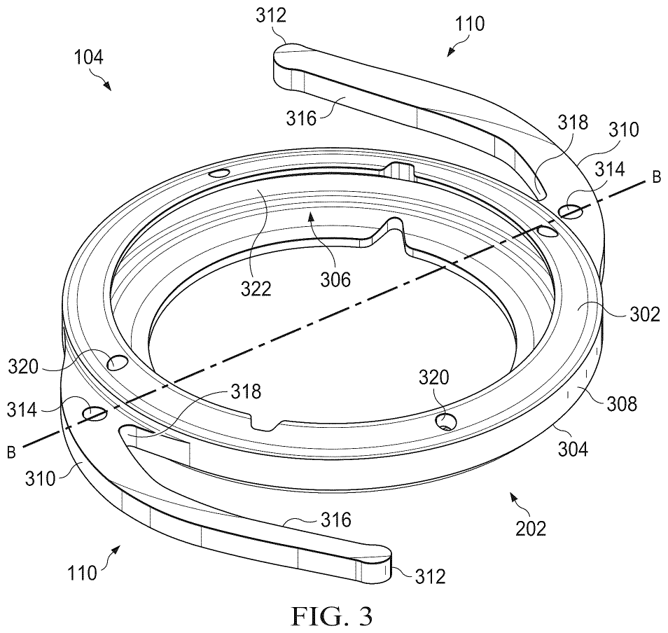

FIG. 3 is an isometric view of an example of the base 104 of FIG. 1, illustrating additional details that may be associated with some embodiments. For example, the ring 202 may be formed with an anterior surface 302, a posterior surface 304, an inner surface 306, and an outer surface 308, one or more of which may be formed as a smooth, continuous surface. The haptics 110 may be coupled to the outer surface 308, as illustrated in the example of FIG. 3. For example, the haptics 110 may be cantilevered on diametrically opposite sides of the ring 202. In more particular examples, the haptics 110 may cach comprise a gusset region 310 and a distal region 312. The haptics 110 may also define an axis across the ring 202 in some embodiments. For example, the gusset region 310 may have one or more openings 314, which may define an axis B-B. An inner surface 316 of the gusset region 310 and the outer surface 308 of the ring 202 can form a junction 318 between the ring 202 and the haptics 110.

The base 104 may additionally or alternatively have one or more openings 320 in one or more of the anterior surface 302, the posterior surface 304, the inner surface 306, and the outer surface 308, as shown in the example of FIG. 3. In some examples, the inner surface 306 may also be formed with a recess 322.

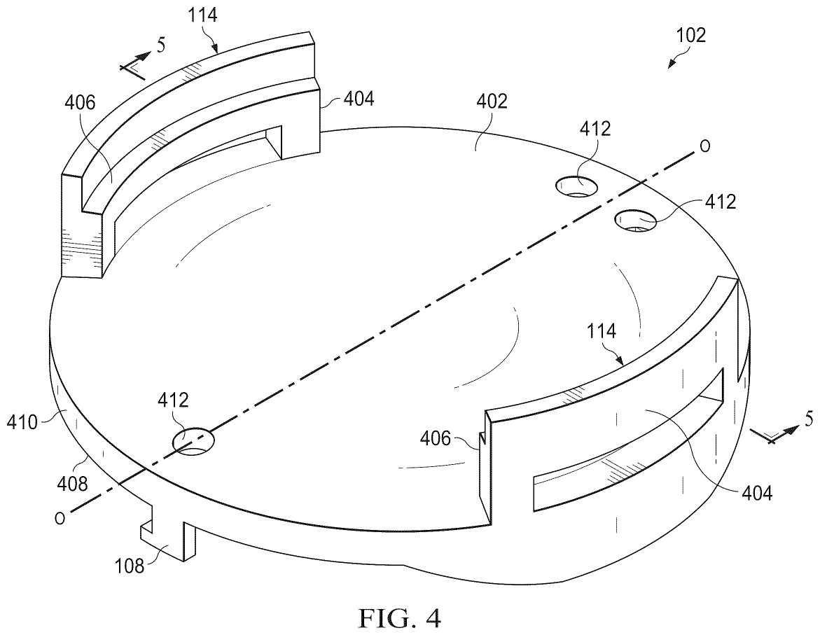

FIG. 4 is an isometric view of the primary optic 102 of FIG. 1, illustrating additional details that may be associated with some embodiments. For example, the optic stabilizers 114 of FIG. 4 may be coupled to an anterior surface 402 of the primary optic 102 and may generally comprise an adjunct support 404 and an optic seat 406. In some examples, the optic seat 406 may comprise or consist essentially of a recess or groove in the adjunct support 404. As illustrated in the example of FIG. 4, some embodiments of the tabs 108 may be coupled to a posterior surface 408 of the primary optic 102. The tabs 108 may be disposed adjacent to an optic edge 410, which generally forms a perimeter surface between the anterior surface 402 and the posterior surface 408.

Additionally, or alternatively, the primary optic 102 may have one or more orientation markers 412, which can identify an orientation axis on the primary optic 102. In the example of FIG. 4, the orientation markers 412 generally comprise holes or dimples on the primary optic 102. As illustrated in the example of FIG. 4, some embodiments of the primary optic 102 may have two orientation markers 412 on one side of the primary optic 102 and one orientation marker 412 on an opposite side. In some embodiments, the orientation markers 412 may facilitate identifying an axis O-O associated with an orientation of the primary optic 102. Additionally, the locking tabs 108 may be aligned with the axis O-O in some examples. The orientation markers 412 may also mark or indicate toricity, which can further facilitate alignment of the primary optic 102, as well as more generally the optical implant 100, in some embodiments.

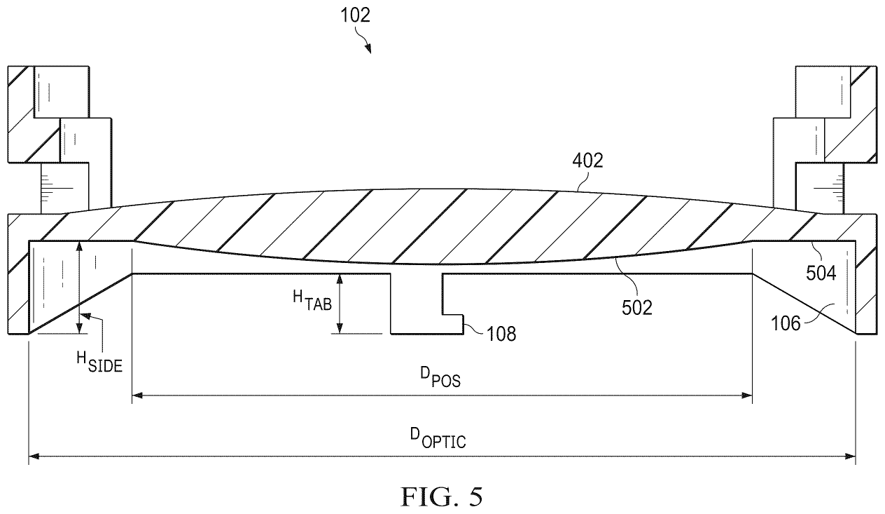

FIG. 5 is a section view of the primary optic 102 of FIG. 4 taken along line 5-5, illustrating additional details that may be associated with some embodiments. For example, an optic portion of the anterior surface 402 may be defined over the optic diameter (DOPTIC) of the primary optic 102. A posterior surface 502 of the primary optic 102 may define a clear posterior optic diameter (Dpos), which may be less than DOPTIc. The posterior side of the primary optic 102 may include a transition region 504 between the posterior surface 502 and the sidewalls 106. The posterior optic diameter (Dpos) may be equal to or greater than a diameter of the inner surface 306 of ring 202 (as shown in FIG. 3). In some embodiments, the posterior optic diameter (Dpos) of the posterior surface 502 may be approximately the same as a diameter of the inner surface 306 of the ring 202 so that the width of the transition region 504 equals the width of the ring 202.

The sidewall 106 of FIG. 5 is configured with a height (HSIDE), and cach tab 108 of FIG. 5 is configured with a height (HTAB) such that the sidewall 106 and tabs 108 cach extend in the posterior direction from the primary optic 102.

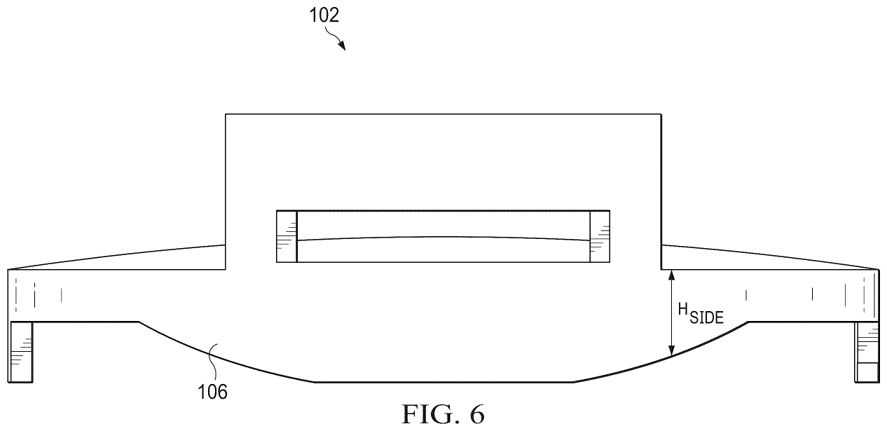

FIG. 6 is a side view of the primary optic 102 of FIG. 4, illustrating additional details that may be associated with some embodiments. For example, the height HSIDE of the sidewall 106 may vary circumferentially, and the sidewall 106 may be tapered or curved to improve usability.

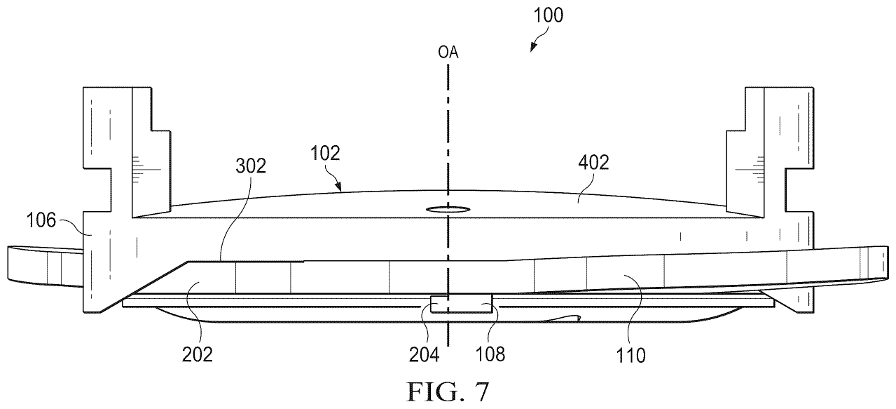

FIG. 7 is a front view of the optical implant 100 of FIG. 1, illustrating additional details that may be associated with some embodiments. As illustrated, the primary optic 102 may be configured with a large optic zone capable of being seated on (instead of seated in) the ring 202. Being positioned on or over the ring 202, the primary optic 102 may be more anteriorly positioned, as well as have a larger anterior surface 402. These features of the primary optic 102 may offer benefits, such as, for example, mitigating undesirable photic phenomena and reducing the prevalence of glint.

For example, the sidewall 106 and the tabs 108 may at least partially overlap the ring 202 to facilitate positioning of the primary optic 102 on the ring 202 and may prevent or substantially reduce decentration and tilt post-surgery. In some embodiments, the height of the sidewall 106 may be less than the thickness of the ring 202 such that the sidewall 106 overlaps a portion of the ring 202. In other embodiments, the height of the sidewall 106 may be equal to or greater than the thickness of the ring 202 so that the sidewall 106 overlaps to extend a distance posterior to the ring 202.

If the primary optic 102 is seated on the base 104 as illustrated in the example of FIG. 7, positioning the extensions 204 posterior to the haptics 110 may secure the primary optic 102 to the base 104. In some embodiments, if the primary optic 102 is seated on the anterior surface 302 of the ring 202, the height of the tabs 108 can ensure that the lateral extensions 204 are generally positioned posterior to the haptics 110. If the lateral extensions 204 are positioned posterior to the haptics 110, rotation of the primary optic 102 in a first direction (e.g., clockwise) relative to an optical axis (OA) can position the lateral extensions 204 posterior and proximate to the haptics 110 such that the primary optic 102 and the ring 202 are connected. In some embodiments, the optic 102 may be rotatable in a first direction until the lateral extensions 204 contact the haptics 110 to secure the primary optic 102 to the base 104.

Conversely, some embodiments of the primary optic 102 may be rotated in an opposite direction (e.g., counterclockwise relative to optical axis OA) to detach the primary optic 102 from the base 104. For example, the lateral extensions 204 may be moved from a position proximate to the haptics 110 to a position that is not proximate to the haptics 110 to that the primary optic 102 and the ring 202 are disconnected, and the primary optic 102 can be separated from the ring 202.

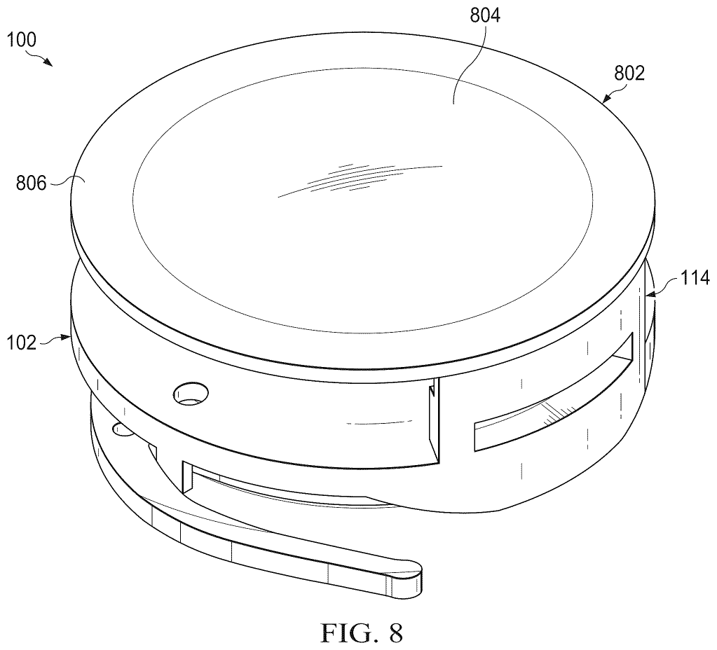

FIG. 8 is an isometric view of another example of the optical implant 100. In the example of FIG. 8, the optical implant 100 further comprise an adjunct optic 802, which may be seated on or otherwise coupled to the optic stabilizers 114 of the primary optic 102. As shown in the example of FIG. 8, the adjunct optic 802 may generally comprise or consist essentially of a central optic 804 and a peripheral optic 806.

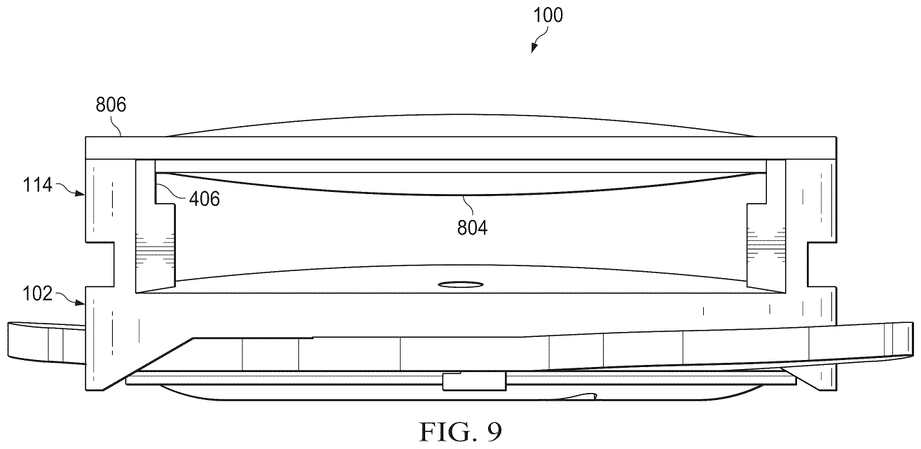

FIG. 9 is a front view of the optical implant 100 of FIG. 8. In the example of FIG. 9, the peripheral optic 806 is supported by the optic stabilizers 114 of the primary optic 102, elevating the central optic 804 relative to the primary optic 102. Additionally, some embodiments of the optic seat 406 may be adapted to receive a portion of the adjunct optic 802. For example, the optic seat 406 of FIG. 9 is configured to receive a portion of the central optic 804, which can further stabilize the adjunct optic 802 relative to the primary optic 102. In some embodiments, the optic stabilizers 114 may be configured to hold an adjunct optic, such as adjunct optic 802, at different distances from the anterior surface 402 of the primary optic 102.

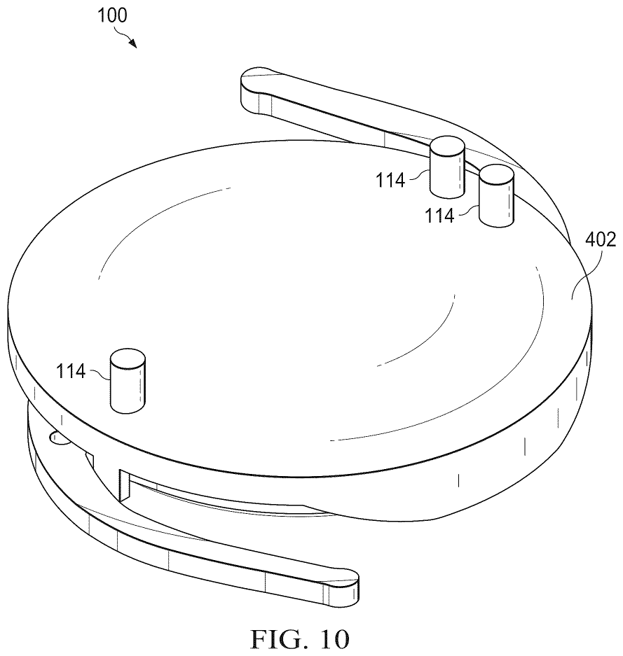

FIG. 10 is an isometric view of another example of the optical implant 100, illustrating various features that may be associated with some embodiments. As illustrated in the example of FIG. 10, some embodiments of the optic stabilizers 114 may comprise a stub or post protruding from the anterior surface 402. The optic stabilizers 114 may also function as orientation markers, such as the orientation markers 412 of FIG. 4.

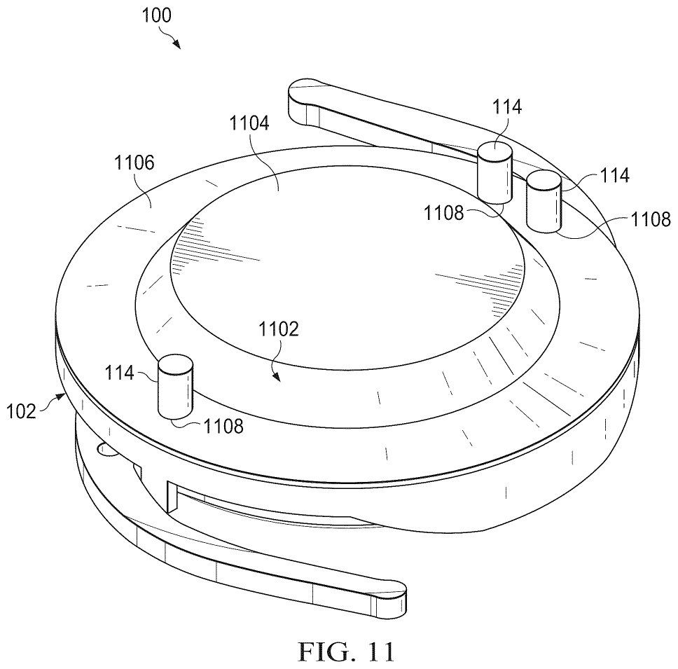

FIG. 11 is an isometric view of another example of the optical implant 100 illustrating additional details that may be associated with some embodiments. In the example of FIG. 11, the optical implant 100 further comprise an adjunct optic 1102, which may be seated on or otherwise coupled to the optic stabilizers 114 of FIG. 10. As shown in the example of FIG. 11, the adjunct optic 1102 may generally comprise or consist essentially of a central optic 1104 and a peripheral optic 1106. In the example of FIG. 11, the peripheral optic 1106 may be supported, at least in part, by the primary optic 102. The adjunct optic 1102 may additionally comprise one or more openings 1108. In some embodiments, the openings 1108 may function as orientation markers, such as the orientation markers 412 of FIG. 4. For example, the optic stabilizers 114 of FIG. 11 may be aligned with the openings 1108 to facilitate orienting the adjunct optic 1102 relative to the primary optic 102. Additionally, the openings 1108 may engage the optic stabilizers 114 of the primary optic 102 to substantially reduce or prevent rotation of the adjunct optic 1102 relative to the primary optic 102.

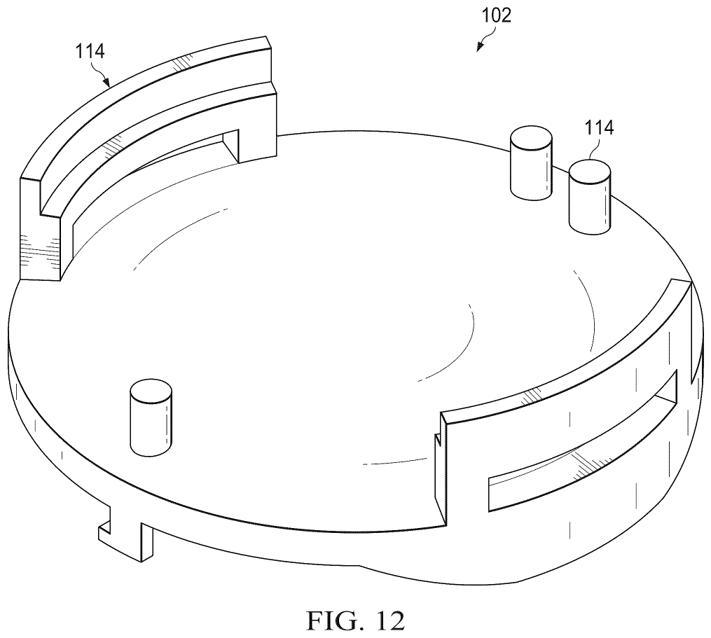

FIG. 12 is an isometric view of another example of the primary optic 102, illustrating additional details that may be associated with some embodiments. For example, some embodiments of the primary optic 102 may comprise various combinations of different types of optic stabilizers. As shown in FIG. 12, for example, the primary optic 102 may comprise a combination of the optic stabilizers 114 of FIG. 1 and FIG. 10.

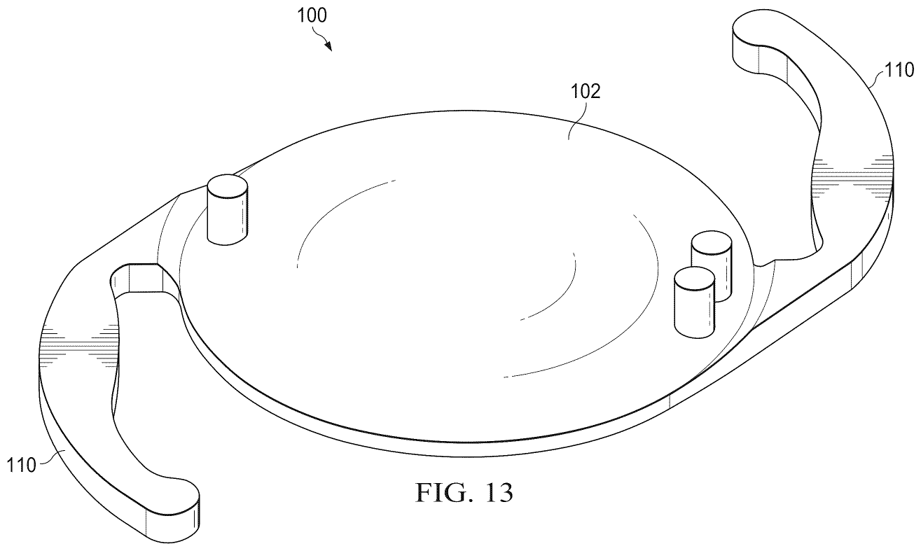

FIG. 13 is an isometric view of another example of the optical implant 100, illustrating additional details that may be associated with some embodiments. For example, some embodiments may have a monolithic structure, as illustrated in FIG. 13. More particularly, in the example of FIG. 13, the haptics 110 may be coupled to or integral with the primary optic 102.

In use, the optical implant 100 may be inserted and positioned in a capsular bag. For example, the optical implant 100 may be implanted by initially delivering the base 104 into a capsular bag in a rolled configuration using an injector (a.k.a., inserter or delivery tube) inserted through a corneal incision, through the capsulorhexis, and into the capsular bag. The base 104 may be ejected from the injector and allowed to unfurl. With gentle manipulation, the haptics 110 can engage the inside of the lens capsule and the ring 202 can be centered relative to the capsulorhexis. The openings 314 and/or the openings 320 may facilitate handling of the base 104, and the axis B-B can indicate an orientation of the base 104. The haptics 110 may be shaped for positioning and stabilizing the base 104 in the capsular bag. The openings 314 can be used to manipulate the base 104.

The primary optic 102 may also be delivered in a rolled configuration using an injector, positioning the distal tip thereof adjacent to the base 104. The primary optic 102 may be ejected from the injector and allowed to unfurl. With gentle manipulation, the primary optic 102 can be centered relative to the capsulorhexis. In some embodiments, the orientation markers 412 can be used to manipulate the primary optic 102 in the capsular bag, for removal of the primary optic 102 from a capsular bag, and to help align the primary optic 102 relative to the base 104. For example, in some embodiments, the orientation markers 412 can be used to align the axis O-O relative to the axis B-B.

Once the primary optic 102 has been delivered and unfurled in the capsular bag, the primary optic 102 may be connected to the base 104. For example, in some embodiments, the primary optic 102 may be connected to the base 104 by first seating the primary optic 102 on the base 104. The sidewalls 106 and the tabs 108 may overlap at least a portion of the base 104 to facilitate the positioning of the primary optic 102 on the base 104 or orienting the primary optic 102 relative to the base 104 during surgery. For example, in some embodiments, the optic 102 may be positioned on the base 104 such that the sidewalls 106 overlap at least a portion of the ring 202. A small force may be applied until the transition region 504 contacts the anterior surface 302 of the ring 202.

If the primary optic 102 is seated on the base 104, the primary optic 102 may be manipulated to generally position the tabs 108 in the junctions 318. For example, in some embodiments, the orientation markers 412 can be used to manipulate the primary optic 102 to position the tabs 108 in the junctions 318. If the tabs 108 are positioned in the junctions 318, rotation of the primary optic 102 relative to optical axis (OA) may position the extensions 204 under the haptics 110. In some embodiments, positioning the extensions 204 under the haptics 110 may cause contact between the extensions 204 and the posterior surfaces of the haptics 110. If the tabs 108 are generally positioned in the junctions 318, a clockwise rotation of the primary optic 102 relative to optical axis (OA) can position the extensions 204 posterior to the haptics 110 such that the extensions 204 contact the posterior surfaces of the haptics 110. If the primary optic 102 is positioned on the base 104, the sidewalls 106 and the tabs 108 can reduce or even prevent tilt and decentration of the primary optic 102.

The adjunct optic 802 may also be delivered in a rolled configuration using an injector, positioning the distal tip thereof adjacent to the primary optic 102. The adjunct optic 802 may be ejected from the injector and allowed to unfurl. With gentle manipulation, the adjunct optic 102 can be coupled to the optic stabilizers 114, such as in the examples described above with reference to FIGS. 8-11.

The systems, apparatuses, and methods described herein may provide significant advantages. For example, in various embodiments, the primary optic 102, the adjunct optic 802, or both, can be adjusted or exchanged while leaving the base 104 in place, either intra-operatively or post-operatively. The optical implant 100 may also improve optical outcomes through rigid alignment of adjunct optics, mitigation of anterior capsule opacification, improving the identification of optical features in vivo during surgery, and reduction of refractive error.

Additionally, or alternatively, various embodiments of the optical implant 100 can reduce surgical complexity, and increase mechanical stability, usability, and efficacy.

While shown in a few illustrative embodiments, a person having ordinary skill in the art will recognize that the systems, apparatuses, and methods described herein are susceptible to various changes and modifications that fall within the scope of the appended claims. Moreover, descriptions of various alternatives using terms such as “or” do not require mutual exclusivity unless clearly required by the context, and the indefinite articles “a” or “an” do not limit the subject to a single instance unless clearly required by the context. Components may also be combined or eliminated in various configurations for purposes of sale, manufacture, assembly, or use.

The claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described in the context of some embodiments may also be omitted, combined, or replaced by alternative features serving the same, equivalent, or similar purpose without departing from the scope of the invention defined by the appended claims.

What is claimed is:

1. An optical implant, comprising:
a posterior optic surface;
an anterior optic surface;
an optic edge between the posterior optic surface and the anterior optic surface;
a locking tab coupled to the posterior optic surface; and
an optic stabilizer coupled to the anterior optic surface.

2. The optical implant of claim 1, wherein the optic stabilizer comprises an angular protrusion from the anterior optic surface.

3. The optical implant of claim 1, wherein the optic stabilizer comprises an adjunct support and an optic seat.

4. The optical implant of claim 1, wherein the optic stabilizer comprises a stub protruding from the anterior optic surface.

5. The optical implant of claim 1, further comprising a base configured to be coupled to the locking tab.

6. The optical implant of claim 5, wherein the base comprises:
a ring having an anterior surface, a posterior surface, an inner surface, and an outer surface; and
a haptic coupled to the outer surface.

7. The optical implant of claim 6, wherein:
the haptic forms a junction with the ring; and
the locking tab is configured to be positioned in the junction.

8. The optical implant of claim 1, further comprising an adjunct optic coupled to the optic stabilizer.

9. An optical implant, comprising:
a base; and
a primary optic, the primary optic comprising:
a posterior optic surface,
an anterior optic surface,
an optic edge between the posterior optic surface and the anterior optic surface,
a sidewall,
a transition region between the posterior optic surface and the sidewall,
locking tabs coupled to the posterior optic surface,
a first optic stabilizer coupled to the anterior optic surface, and
a second optic stabilizer coupled to the anterior optic surface;
wherein the locking tabs and the sidewall are configured to overlap the base.

10. The optical implant of claim 9, wherein the second optic stabilizer is disposed opposite the first optic stabilizer.

11. The optical implant of claim 9, wherein:
the first optic stabilizer comprises a first adjunct support and a first optic seat;
the second optic stabilizer comprises a second adjunct support and a second optic seat; and
the second adjunct support is disposed opposite the first adjunct support.

12. The optical implant of claim 9, wherein the base comprises:
a ring having an anterior surface, a posterior surface, an inner surface, and an outer surface; and
a pair of haptics coupled to the outer surface.

13. The optical implant of claim 12, wherein:
the haptics form junctions with the ring; and
the locking tabs are configured to be positioned in the junctions.

14. The optical implant of claim 9, further comprising an adjunct optic coupled to the first optic stabilizer and the second optic stabilizer.

15. The optical implant of claim 13, wherein:
the base defines a first axis;
the primary optic defines a second axis; and
the locking tabs are aligned with the second axis;
wherein positioning the locking tabs in the junction aligns the second axis with the first axis.

16. The optical implant of claim 15, wherein:
the base comprises openings that define the first axis; and
the primary optic further comprises orientation markers that define the second axis.

17. A method for assembling an optical implant, the method comprising:
providing a base, a primary optic, and an adjunct optic, wherein the primary optic comprises a locking tab, a sidewall, and an optic stabilizer;
positioning the primary optic on the base so that sidewall overlaps at least a portion of the base;
rotating the primary optic relative to the base to couple the locking tab to the base; and
coupling the adjunct optic to the optic stabilizer.

18. The method of claim 17, wherein:
the base comprises a ring having an anterior surface, a posterior surface, an inner surface, and an outer surface;
a haptic coupled to the outer surface;
the haptic forms a junction with the ring; and
rotating the primary optic positions the locking tab in the junction.

19. The method of claim 17, wherein:
the base further comprises openings that define a first axis;
the primary optic further comprises orientation markers that define a second axis;
the locking tab is aligned with the second axis; and
rotating the primary optic aligns the first axis and the second axis.
